(19) United States
(12) Patent Application Publication
(10) Pub. No.: US 2019/0254866 A1
(43) Pub. Date: Aug. 22, 2019

(54) Cooling Device

(71) Applicant: THE WHITELEY CLINIC LIMITED, Guildford (GB)

(72) Inventors: Mark Whiteley, Guildford (GB); Jaya Nemchand, Guildford (GB)

(21) Appl. No.: 16/345,618

(22) PCT Filed: Oct. 26, 2017
(86) PCT No.: PCT/GB2017/053228

§ 371 Date: Apr. 26, 2019

(30) Foreign Application Priority Data
Oct. 28, 2016 (GB) 1618255.2

(51) Int. Cl. A61F 7/12; A61M 19/00

(52) U.S. Cl. CPC A61F 7/12; A61F 2007/0075; A61M 19/00

(57) ABSTRACT

A cooling device for cooling a subject's tissue or organ surface, the device comprising a handle and a head. The head comprises an electrical cooling element for cooling the subject's tissue or organ surface. The device also comprises a controller configured to: receive the initial temperature of the subject's tissue or organ surface; determine a cooling temperature based on the initial temperature of the subject's tissue or organ surface and a target temperature; and control the cooling element to generate the cooling temperature. The invention also provides a separate head and handle. The invention also provides a method of controlling the cooling device.

### FIELD

The present invention relates to cooling devices and components thereof, and particularly, although not exclusively, to body surface cooling devices for cooling a region of a subject's body, be that internal or external, while treatment is taking place or prior to piercing it with a needle, scalpel, or other sharp object. The invention is especially concerned with reducing the temperature of a subject's skin or other area of the body prior to giving an injection or taking a blood sample, for example. The present invention also relates to methods of cooling a region of a subject's body surface using the cooling device prior to, or during, any treatment.

### BACKGROUND

A great many medical and cosmetic interventions require the passage of a needle or similar device into or through the skin. For example, it is necessary for a hypodermic needle (i.e. a hollow needle) to pierce the skin of a patient to a sufficient depth to allow a medicament to be administered by an injection. It will be appreciated that the term injection typically encompasses intradermal (ID), intravenous (IV), intramuscular (IM), and subcutaneous (SC) administration, and the location of the depth to which the needle is inserted will depend upon the type of injection. Injections are amongst the most common healthcare procedures, with at least 16 billion administered in developing and transitional countries each year. 95% of injections are administered in curative care, 3% are for immunization, and the rest for other purposes, such as blood transfusions.

It will be appreciated that a hypodermic needle will also need to pierce the skin for a sample, such as blood, to be taken. Alternatively, a cannula may be used to administer and remove fluids instead of a syringe. A cannula generally comprises a short catheter (a few centimetres long) which is inserted through the skin into a vein. Usually, a flexible plastic cannula is provided mounted over a metal trocar. The tip of the trocar and cannula may pierce the skin and be introduced into the vein; the cannula may then be advanced inside the vein over the trocar to the appropriate position and secured. The trocar is then withdrawn and discarded. Medicament can then be delivered, or blood samples may be drawn directly from the patient, once the cannula has been inserted.

Finally, some procedures may involve solid needles, such as for electrolysis. In all of the above cases, the passage of a needle or similar devices through the tissue surface, such as the skin or oral mucosa (during dental procedures) is medically invasive and inherently causes pain. Specifically, in the case of skin, the pain receptors (nociceptors) in the dermis are stimulated when a needle or device passes into the dermis. Accordingly, pain relief may be required. The layer under the dermis, the subcutaneous fat, tends to lead to lesser observed pain when needles are passed into this area. However, if a substance that is an irritant is injected into this fat layer then pain can often result.

It is noted that since needles are often used to apply a small puncture wound to the body, with varying degrees of pain inherent to this, fear of needles is a common phobia. Numerous techniques are commonly used in an attempt to reduce the discomfort felt by the subject. For instance, a clinician may rub the tissue area prior to inserting a needle, utilising the “gate theory”, in an attempt to reduce the pain. Alternatively, distraction techniques and topical local anaesthetics, usually left for upwards of 30 minutes, may be used.

Laboratory and clinical research have shown that cooling the surface of the skin to less than 10° C. reduces the pain felt when the skin is stimulated by the passage of a needle or when other painful stimuli are applied thereto. It has been found that skin that has hair tends to more susceptible to anaesthesia by cooling and the effect lasts longer when the cooling is removed. Accordingly, ice is sometimes applied to the skin to reduce pain. The ice either needs to be contained in a sealed container or will lead to wetness as the ice melts. Moreover, the ice itself may cause damage to the skin.

In addition to these problems, there are also a number of heat treatments used in curative and aesthetic medicine that can cause damage if the heat spreads to a non-target area. For example, new approaches to curing excess underarm sweating rely on applying microwave radiation to the body. The microwaves are intended to penetrate the skin and cause water in cells beneath the skin to become excited (i.e. heated) and irreparably damaged. While damaging the cells beneath the skin is intentional, it is important that the outer layer of skin is not damaged by the heat. It can be difficult to actively heat one area while preventing the heat spreading to a non-target area.

A number of cooling devices have also been developed. Commonly these devices are in one piece. Additionally, other devices, such as a canister comprising a vapour cooling spray are entirely disposable and need to be constantly replaced, leading to an increase in cost.

The invention arises from the inventor's work in attempting to address the problems associated with the prior art.

### SUMMARY

According to a first aspect of the present invention, there is provided a cooling device for cooling a subject's tissue or organ surface, the device comprising:
a handle;
a head comprising:
   an electrical cooling element for cooling the subject's tissue or organ surface; and
a controller configured to receive an initial temperature of the subject's tissue or organ surface; determine a cooling temperature based on the initial temperature of the subject's tissue or organ surface and a target temperature; and control the cooling element to generate the cooling temperature.

Advantageously, the cooling device provides a means to cool a subject's tissue or organ surface such that a needle or similarly sharp instrument can be inserted into the subject's tissue or organ surface painlessly.

According to a second aspect of the present invention, there is provided a head for a cooling device for cooling a subject's tissue or organ surface, the head comprising:
an electrical cooling element for cooling a subject's tissue or organ surface; and
an interface for coupling the head to a handle of a cooling device, such that the head can communicate with the handle.

Advantageously, the head can be discarded and another used in its place for a different patient, or for a different part of the body, without discarding the whole device to which it is attached.

According to a third aspect of the present invention, there is provided a handle for a cooling device for cooling a subject's tissue or organ surface, the handle comprising:
an interface for coupling the handle to a head of the cooling device, such that the handle can communicate with the head; and
a controller configured to:
   receive an initial temperature of the subject's tissue or organ surface;
   determine a cooling temperature based on the initial temperature of the subject's tissue or organ surface and a target temperature; and
   control the cooling element to generate the cooling temperature.

Preferably, the head of the second aspect and/or the handle of the third aspect are attached together to form the cooling device of the first aspect. Preferably, the cooling device comprises a fixing means for removably attaching the head to the handle. Preferably, the fixing means comprises a hook disposed on one of the head and the handle and a groove disposed on the other one of the head and the handle. Alternatively, the fixing means comprises pins and corresponding receiving holes, or clips. Preferably, the controller is disposed in the handle.

The head may be integrally formed with the handle, and the head comprises a removable non-porous layer between the cooling element and the subject's tissue or organ surface. Even more preferably, the non-porous layer comprises an adhesive.

Preferably, the head is rotatable with respect to the handle, such that the head can contact the subject's tissue or organ surface while the handle is held away from the subject's tissue or organ surface. Preferably, the head is rotatable from about −90° to about +90° to the longitudinal axis of the handle.

The controller is preferably configured to determine a cooling time based on the initial temperature of the subject's tissue or organ surface, the target temperature, and the cooling temperature.

The cooling device preferably comprises a temperature sensor for measuring the temperature of the subject's tissue or organ surface.

Preferably, the head further comprises a fixing means for removably attaching the head to the handle. Even more preferably, the fixing means comprises a locking mechanism for fixing the angle of the head with respect to the handle such that the applying pressure to the handle applies pressure to the head.

Preferably, the head comprises at least one fin for dissipating heat generated by the cooling element.

Preferably, the shape of the cooling element substantially corresponds to the shape of the head. It will be appreciated that the size and shape of the head used will depend on the target site on the subject's body, and the nature of the procedure being carried out thereon.

A “subject” may be a vertebrate, mammal, or domestic animal. Hence, the cooling device may be used to treat any mammal, for example livestock (e.g. a horse), pets, or may be used in other veterinary applications. Most preferably, however, the subject is a human being.

The head is preferably annular defining an aperture, and through which a medical instrument, such as a needle, may be passed. The aperture formed by the annular head may be any shape, size and/or diameter to achieve the desired technical effect. Accordingly it can be, but is not limited to, a circular shape, whose dimensions are sufficient to accommodate a needle to be passed therethrough. The aperture can be square, rectangular, triangular, or diamond-shaped, for example.

In another embodiment, the head may comprise one or more spaced-apart curvilinear arms which mutually define an open-ended aperture. The aperture may, for example, have a V-shaped profile or U-shaped profile. This provides the user with unimpeded visibility of the target area.

Preferably, the head comprises a window in the cooling element for allowing light to pass through the head to the subject's tissue or organ surface. The window may comprise a transparent material such as glass, Perspex, or plastic. Even more preferably, the window comprises an air gap. Advantageously, the aperture or window acts as a guide via which the user can direct the head to the target zone on the subject's tissue or organ surface.

Preferably, the cooling element is configured to cool a subject's tissue or organ surface, when activated, to a temperature of between −30° C. and 15° C. More preferably, the cooling element is configured to cool the subject's tissue or organ surface to a temperature of between −25° C. and 10° C. Most preferably, the cooling element is configured to cool the subject's tissue or organ surface to a temperature of between −20° C. and 5° C. Still more preferably, the cooling element is configured to cool the subject's tissue or organ surface to a temperature of between −15° C. and 0° C. Most preferably, the cooling element is configured to cool the subject's tissue or organ surface to a temperature of between −10° C. and −5° C. Preferably, the cooling element is configured to cool the subject's tissue or organ surface to a temperature below 5° C., 0° C., or −5° C.

Preferably, the cooling element comprises a thermoelectric device using the Peltier effect. More preferably, layers of the thermoelectric device comprise bismuth and copper.

Preferably, the upper surface of the head comprises at least one vent for dissipating heat generated by the cooling element.

Preferably, at least the lower surface of the head is fabricated from an expandable material adapted to expand and contract with thermal expansion and/or contraction of the cooling element. More preferably, the lower surface is fabricated from a highly thermally conductive material to maximise thermal energy transfer from the cooling element to the subject's tissue or organ surface to which it is applied. Even more preferably, the lower surface is fabricated from fine gauge stainless steel, other metals, alloys or a similar non-metal material such that the lower surface is sufficiently durable to withstand sudden temperature changes.

Preferably, the lower surface of the head is substantially flat. Alternatively, the lower surface is either concave or convex or a combination of the two. For example, when treating significantly concave areas of the subject's tissue or organ surface, a convex surface is desirable in order that as much of the surface as possible can engage the subject's tissue or organ surface to provide effective contact with the subject's tissue or organ surface. Conversely, when treating significantly convex areas of the subject's tissue or organ surface, a concave surface is desirable.

Preferably, the head defines a hollow cylinder, and preferably the cooling element is disposed on the outer surface of the hollow cylinder such that the inside of a cylindrically-shaped vessel can be cooled.

Even more preferably, the inner surface of the hollow cylinder comprises at least one vent for dissipating heat generated by the cooling element.

Preferably, the head is disposable. Preferably, the head comprises a removable sterile cover.

Thus, preferably the target temperature is between −30° C. and 15° C., more preferably between −25° C. and 10° C., more preferably between −20° C. and 5° C., still more preferably between −15° C. and 0° C., and most preferably between −10 degrees Celsius and −5° C. Preferably, the target temperature is below 5° C., 0° C., or −5° C. Preferably, the controller is configured to vary the cooling temperature.

Preferably, the controller is configured to turn off the cooling element when the target temperature is reached. Alternatively, the controller is configured to calculate a cooling time, and turn off the cooling element after the cooling time. Even more preferably, the handle comprises a user input for selecting a subject's body part to be cooled, and the controller is configured to determine the cooling temperature based on the selected body part.

Preferably, the controller is configured to optimise the cooling temperature based on the cooling time, temperature of the subject's tissue or organ surface, and target temperature.

Preferably, the handle comprises a power source. More preferably, the power source is a battery. Alternatively, the power source is a power adapter for coupling the cooling device to a mains supply.

Preferably, the handle comprises a display for displaying at least one of: the remaining time before the target temperature is reached, the current temperature of the subject's tissue or organ surface, and an indication of whether the target temperature is reached. Additionally or alternatively, the handle comprises an alarm for indicating whether the target temperature is reached.

Preferably, the handle comprises a thermally insulating material.

Preferably, the handle comprises a catheter.

According to a fourth aspect of the present invention, there is provided a method of cooling a subject's tissue or organ surface, the method comprising:
receiving the initial temperature of the subject's tissue or organ surface;
determining a cooling temperature based on the initial temperature of the subject's tissue or organ surface and a target temperature; and
controlling a cooling element to generate the cooling temperature.

Preferably, the initial temperature of the subject's tissue or organ surface is received from a temperature sensor. Alternatively, the initial temperature of the subject's tissue or organ surface is received from a storage means.

The subject's tissue or organ surface which is cooled may be internal, for example a mucous membrane inside the mouth, vagina, nasal cavity or anus, or the outer surface of an internal organ. Preferably, the subject's tissue or organ surface is external, for example any part of the skin or the eye. Advantageously, the device, head and handle may be used to reduce the temperature of at least a region of a subject's tissue or organ surface, be that internal or external, prior to piercing the region with a needle, scalpel or other sharp instrument.

Preferably, the target temperature is between −30° C. and 15° C. More preferably, the target temperature is between −25° C. and 10° C. Most preferably, the target temperature is between −20° C. and 5° C. Still more preferably, the target temperature is between −15° C. and 0° C. Most preferably, the target temperature is between −10 degrees Celsius and −5° C. Preferably, the target temperature is below 5° C., 0° C., or −5° C.

Preferably, the method comprises varying the cooling temperature.

Preferably, the method further comprises receiving an indication of a subject's body part to be cooled, and the determining a cooling temperature comprises determining a cooling temperature based on the initial temperature of the subject's tissue or organ surface, the target temperature and the body part.

Preferably, the method comprises determining a cooling time and optimising the cooling temperature based on the cooling time, initial temperature of the subject's tissue or organ surface, and target temperature.

Preferably, the method comprises turning off the cooling element when the target temperature is reached. Alternatively, the method comprises turning off the cooling element after the cooling element has been activated for the duration of the cooling time.

All features described herein (including any accompanying claims, abstract and drawings), and/or all of the steps of any method or process so disclosed, may be combined with any of the above aspects in any combination, except combinations where at least some of such features and/or steps are mutually exclusive.

### BRIEF DESCRIPTION OF THE FIGURES

Embodiments of the present invention will now be described, by way of example only, with reference to the accompanying drawings, in which: Figure is is a side view of a first embodiment of a cooling device comprising a handle and a head;

FIG. 1b is a top view of the cooling device shown in FIG. 1a;

FIG. 2 is a bottom view of one embodiment of the head of the cooling device;

FIG. 3 is a bottom view of a second embodiment of the head;

FIG. 4 is a perspective view of a mechanical interface between the head and the handle of the cooling device;

FIG. 5 is a side view of a second embodiment of the cooling device;

FIG. 6 is a system diagram of a cooling device according to an embodiment;

FIG. 7 is a perspective view of an embodiment of a cooling element within the head of the cooling device; and

FIG. 8 is a flow diagram illustrating a method of cooling a tissue or organ surface according to an embodiment.

### DETAILED DESCRIPTION

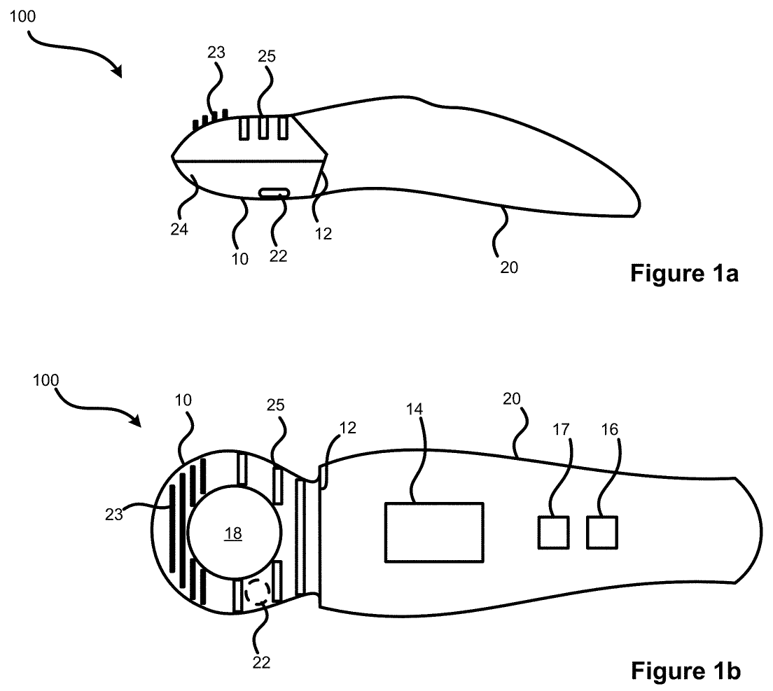

A cooling device 100, also known as a cryoanaesthesia device, shown in FIG. 1, for use in reducing the temperature of a subject's tissue or organ surface prior to piercing it with a needle or other sharp instrument, such as a scalpel. The cooling device 100 can also be used to cool a part of a tissue or organ surface while another part of the tissue or organ surface is being actively heated. This prevents the heat from damaging unintended areas of the body tissue. The tissue or organ surface can be internal, such as the oral mucosal membrane (for use by dentists), a surface of an organ such an underside of the skin, or the inner membrane of the rectum or vagina, or external, such as the skin or the surface of the eye.

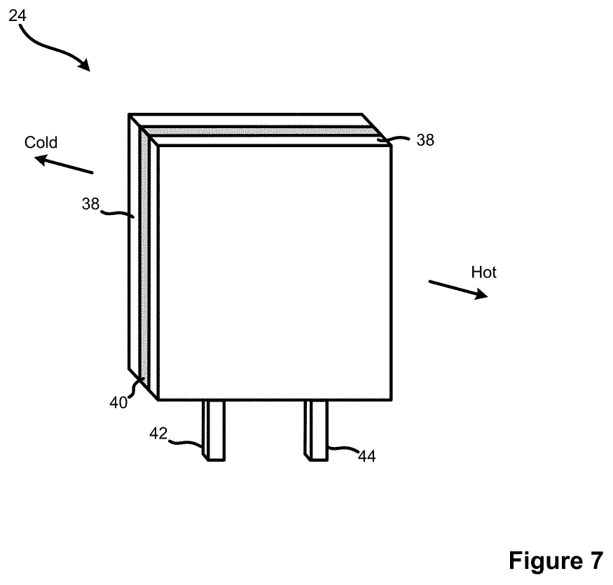

The device 100 comprises a head 10 connected to a handle 20. While the handle 20 shown in the Figures is an ergonomic design, in some embodiments the head 10 is connected to a catheter such that it can be inserted into the body. Generally, the head comprises a cooling element 24 (shown in detail in FIG. 7) and the handle 20 comprises control components for controlling the cooling element 24. The cooling element 24 according to an exemplary embodiment is a thermoelectric device which utilises the Peltier effect, described in more detail with reference to FIG. 7. It would be appreciated that the cooling element 24 in other embodiments is any reusable is cooling means. The handle 20 is ergonomically designed such that the device 100 is operable with one hand.

When working with humans or animals, it is essential to maintain a sterile environment. Particularly, if an instrument is not sterile, the bacteria that may have collected on the instrument can be transferred to a tissue or organ surface, and then infect a wound when the tissue or organ surface is punctured by a needle. Therefore, medical instruments need to be easy to sterilise, or designed for one-time use. Sterilising instruments typically involves heating the instrument using steam in an autoclave. Clearly, this is not appropriate for instruments having electrical components. Methods of sterilization also include using gamma radiation and Ethylene Oxide (EtO) treatment, but these are costly and labour-intensive.

Therefore, in one embodiment, the present invention provides a cooling device 100 having a disposable head 10. This prevents wastage of the handle 20 which has not come into contact with another human or animal.

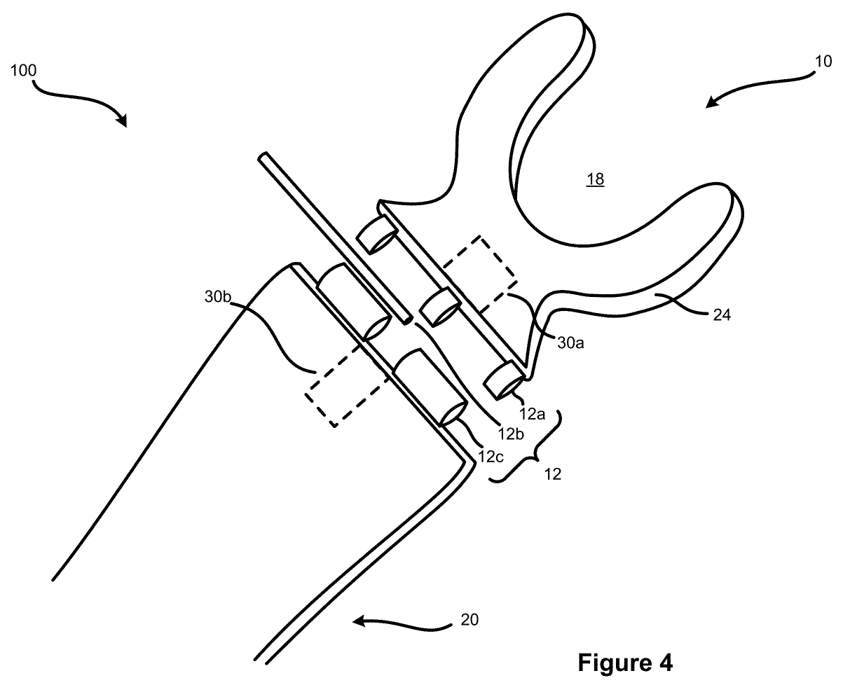

The head 10 is coupled to the handle 20 at a mechanical interface 12, which will be described with reference to FIG. 4. The mechanical interface 12 allows the head 10 to rotate relative to the longitudinal axis of the handle 20. Preferably the mechanical interface 12 allows the head 19 to rotate between 0° and about +/−90° to its longitudinal axis. However, it would be readily understood that for some difficult to reach parts of the body, it is advantageous to have the head 10 back towards the handle 20 such that it faces the user. Here, the head 10 rotates between about −180° and about +180° to the longitudinal axis. It would be apparent in practice that the head 10 cannot rotate a full 180° to the longitudinal axis as the handle 20 will block its traverse. In some embodiments, the head 10 is coupled to the handle 20 such that it can twist (i.e. rotate in parallel to the longitudinal axis) and rotate (i.e. perpendicularly to the longitudinal axis) relative to the handle 20.

The mechanical interface 12 includes a locking mechanism (not shown) for fixing the angle of the head 10 relative to the handle 20. The locking mechanism may be a mechanical switch connected to a member to block the traverse of the head 10, or a resilient member such as a coil spring or cushion. Therefore, once the appropriate angle has been achieved, it can be fixed so that pressure can be applied to the head 10 through the handle 20.

The head 10, provided separately to the handle 20, is encased in a sterile air-tight package prior to use. The cooling element 24 is disposed at a lower portion of the head 10. The lower portion of the head 10 is the side of the head 10 closest to the subject's tissue or organ surface when the cooling device wo is used. The tissue or organ surface may be, for example, the subject's skin, mucous membrane inside the mouth (oral mucosa) or genital region, anus, nasal cavity, or organs (internal or external) such as the eye or lungs.

As shown in FIG. 1, an upper portion of the head 10 comprises at least one vent 25 for dissipating heat generated by the cooling element 24. The upper portion of the head 10 may also comprise fins 23 for increasing the surface area of the head 10, and therefore improve heat dissipation.

In some embodiments, the head 10 also includes a temperature sensor 22 for sensing the temperature of the tissue or organ surface. The temperature sensor 22 is any known means for detecting the temperature of an object, converting the temperature into temperature information and transmitting the temperature information electronically. For example, the temperature sensor 22 is a thermocouple. In other embodiments, the temperature sensor 22 is one of a Negative Temperature Coefficient thermistor, a Resistance Temperature Detector and a semi-conductor-based sensor. The temperature sensor 22 is formed on a lower side of the head 10, such that it is in close contact with the subject's tissue or organ surface when the cooling device 100 is in use.

In still other embodiments, the temperature sensor 22 is not necessary. Here, the body or organ tissue's initial temperature is assumed to be within a normal range known in the art. For example, the skin's surface temperature on a healthy human is about 34 degrees Celsius. This initial temperature, or temperature range from which the most conservative value is chosen for safety, may be received manually from a user input, or received from a storage means. An algorithm executed by a processor or controller in the cooling device 100 is used to determine the length of time and cooling temperature necessary to cool the tissue or organ surface to a target temperature from the initial temperature. The cooling process is determined to be complete when the determined time expires.

The cooling side of the cooling element 24 may form part of the outer surface of the head 10. Alternatively, the head 10 may comprise a housing, with the cooling element 24 disposed inside the housing.

When the cooling element 24 does not form the outer surface of the head 10, at least the lower surface of the head 10 is fabricated from an expandable material adapted to expand and contract with thermal expansion and/or contraction of the cooling element 24. The material is highly thermally conductive to maximise thermal energy transfer from the cooling element 24 to the tissue or organ surface to which it is applied. For example, the lower surface is fabricated from fine gauge stainless steel, other metals, alloys or a similar non-metal material such that the lower surface is sufficiently durable to withstand sudden temperature changes.

In some embodiments, the lower surface of the head 10, be it formed of the cooling element itself 24 or a separate housing layer, is substantially flat. Alternatively, the lower surface of the head 10 is either concave or convex or a combination of the two. For example, when treating significantly concave areas of the tissue or organ surface, a convex surface is desirable in order that as much of the surface as possible can engage the tissue surface to provide effective contact with the tissue surface. Conversely, when treating significantly convex areas of the tissue or organ surface, a concave surface is desirable.

In the embodiment shown in FIG. 1b, the head 10 is annular. In other words, the head 10 forms the outside of an opening 18 through which a needle can be inserted. The opening 18 effectively provides a viewing area through which the target site on the subject's tissue or organ surface can be viewed, be that a vein or hair follicle etc., and also provides access for a needle. The cooling element 24 covers a significant proportion of the area of the head 10 which makes contact with tissue or organ surface. In use, the cooling device 10 may be removed from the tissue or organ surface prior to injection, or the needle may be inserted directly through the opening 18. The opening 18 and cooling element 24 are arranged such that nerves surrounding a target are cooled, but the target itself is not cooled. For example, a thread vein would be difficult to inject into if it had itself been cooled. However, by targeting the nerves around the vein, the vein remains coloured and therefore easy to identify.

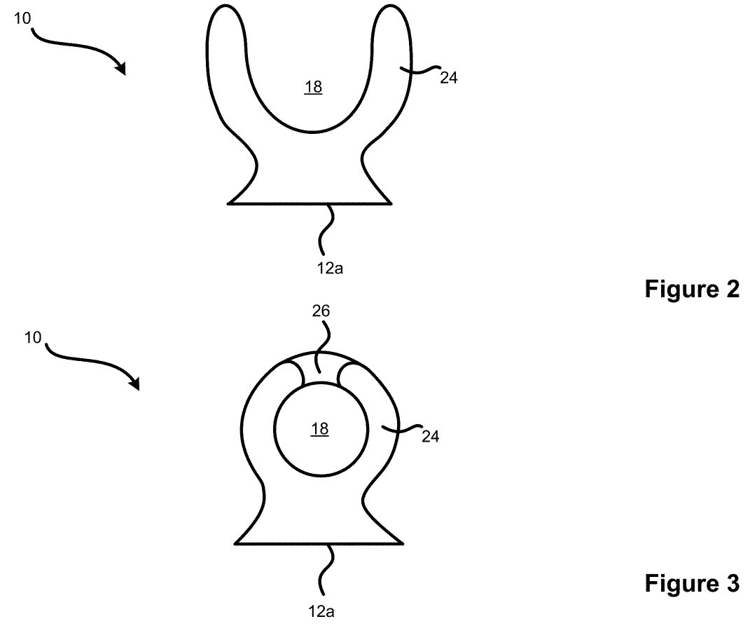

Head 10 shapes according to alternative embodiments are shown in FIGS. 2 and 3. In the embodiment of FIG. 2, the head 10 does not form a circular aperture 18 at its centre. Instead, the aperture, or opening 18 is semi-circular created by two spaced apart curvilinear arms. This provides the user with unimpeded visibility of the target area.

An alternative approach to solve the same problem of improving visibility when using the device 100, without substantially reducing the cooling area, is shown in FIG. 3. Here, a transparent window 26 is arranged to allow light to pass through the cooling element 24. The window 26 is arranged at the distal end of the head 10. In other embodiments, the window is disposed at the side or rear of the head 10. The window 26 is, for example, made of any suitable transparent material such as Perspex, plastic or glass. Replacing the window 26 with an air gap would effectively provide the design shown in FIG. 2.

Alternatively to the shapes shown here, bespoke heads 10 tailored to specific body areas and injection sites may be used. For example, the opening 18 may be “V”, “U” or “C” shaped to ensure accurate cooling.

Furthermore, the head 10 may be cylindrical so that it can easily be inserted into the body. Particularly, a cylindrical shape is useful when the cooling device 10 is used to cool the inner surface of a blood vessel or other tubular structure. The cylindrical head 10 may be a hollow cylinder, i.e. having an opening 18 at its central region along the longitudinal axis of the head 10. In these embodiments, the cooling element 24 may fully surround the outer surface of the head 10, while heat is dissipated into the head's central region (i.e. the opening 18). The opening 18 may comprise vents and/or fins for dissipating heat energy away from the tissue or organ surface more effectively.

Returning to FIG. 1b, the handle 20 comprises a display 14. The display 14 is, for example, an LCD display. In alternative embodiments, the display 14 is an LED display or an OLED display. The display 14 shows whether the tissue or organ surface (for example, skin) is at the appropriate temperature for injection. In other words, the display 14 provides an indication that the nerves around a target area have been sufficiently cooled such that the target area is numb. The display 14 may display the tissue or organ surface temperature directly, or a message indicating that the target temperature has been reached. The display 14 may flash to indicate the target temperature has been reached. In some embodiments, the display 14 displays the time left before the tissue or organ surface will reach the target temperature. The target temperature is typically between −10° C. and −5° C. In other words, the target temperature is set to numb nerves, without causing damage to the tissue or organ surface, which may be internal or external to the body. Additionally, the target temperature can be set to keep the tissue or organ surface at a relatively constant temperature while surrounding tissue is heated.

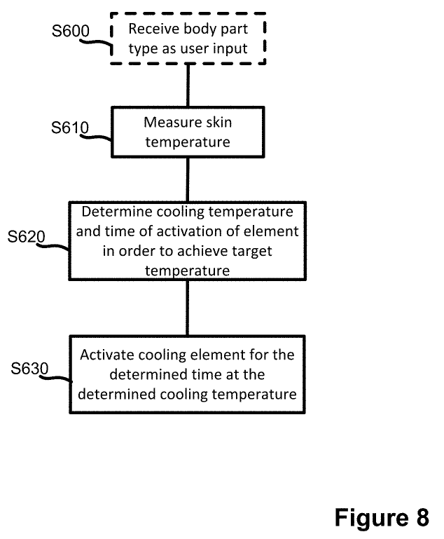

The handle 20 of the cooling device 100 may also include a sound generator and/or an LED to indicate whether the desired target temperature has been reached. The handle 20 includes an on/off switch 16 for turning the device 100 on and starting the process described with reference to FIG. 8. The cooling element 24 deactivates automatically when the target temperature is reached. If the device 100 is left switched on, the cooling element 24 reactivates when the tissue or organ surface temperature rises above a threshold temperature greater than the target temperature, in order to keep the temperature of the tissue or organ surface within a predetermined range.

The handle 20 also includes a user input 17 for selecting the body part having the tissue or organ surface which the user intends to cool. For example, the skin covering the thighs reacts differently to the skin around the eyes or the mucous membrane inside the mouth (oral mucosa), or the outer surface of an internal organ. The nerves may be denser, or buried deeper in the thighs, and so that the cooling element 24 needs to cool for longer. It may be the case that the area of skin is less sensitive, and so the cooling element 24 can output a lower cooling temperature in order to achieve the target temperature without causing discomfort.

The cooling device wo may be used by a body piercer, for example, to reduce the pain cause by piercing a nostril. The cooling device wo may also be used by a dentist, for example, to reduce the pain caused by injecting anaesthetic into a gum. The cooling device wo may also be used, for example, by a cosmetic surgeon to reduce the pain caused by injecting collagen into lips. Furthermore, the cooling device wo may be used with an electrolysis treatment. Electrolysis is the procedure for hair follicle removal using a fine, sterile and disposable needle to permanently destroy the follicle's ability to reproduce. This is achieved by destroying the hair making cells in the follicle wall of each hair, using chemical or heat energy. The cooling device wo is applied to the skin before the fine needle is inserted in order to reduce the pain sensed when the needle is inserted.

The user input 17 shown in FIG. 1b is a manual selector switch. However, in other embodiments, the display 14 is a touchscreen that provides a user input.

In some embodiments, the user input 17 provides a means to manually increase or decrease the temperature of the cooling element 24. The temperature of the cooling element 24 is also known as the cooling temperature.

An example of a mechanical interface 12 for coupling the head 10 to the handle 20 is shown in FIG. 4. The interface 12 provides a means for attaching the head 10 to the handle 20, while allowing the head 10 to rotate relative to the handle 20. Primarily, this allows the head 10 to be swapped for a sterile head 10 between patients, or for a different type of head 10 for a different tissue or organ surface shape. Additionally, allowing the head 10 to rotate allows the handle 20 to be held comfortably away from the patient, while the head 10 remains in contact with the tissue or organ surface across its lower surface area.

A locking unit (not shown) fixes the angle of the head 10 with respect to the handle 20 once the desired angle has been achieved. This allows pressure applied to the handle 20 to be transmitted to the head 10 without causing further rotation. For example, the locking unit is a slider that slides over the interface 12 to prevent rotation. Additionally or alternatively to the locking unit, the interface 12 may be biased, for example using a coil spring such that it is resilient to movement.

In the embodiment shown in FIG. 4, the interface 12 includes rings 12a disposed on the proximal end of the head 10. The interface 12 also includes rings 12c disposed on the distal end of the handle 20. The rings 12a of the head 10 and rings 12c of the handle 20 have a corresponding internal diameter. A pin 12b is used to pass through each of the rings 12a, 12c, thereby rotatably coupling the head 10 to the handle 20. The pin 12b can be pushed or pulled out of the rings 12a, 12c, so that the head 10 can be detached from the handle 20 without causing damage to the handle 20.

In other embodiments, the interface 12 comprises a universal joint, such that the head 10 can rotate and twist relative to the handle 20.

The rotatable aspect of the head 10 is not essential, and in other embodiments any well-known mechanical interface may be used to couple the head 10 to the handle 20 while allowing the head 10 to be later removed without causing damage to the handle 20. For example, one of the head 10 and handle 20 may include deformable hooks, and the other of the head 10 and handle 20 may include grooves or holes to receive the deformable hooks. The hooks are deformable such that they can be depressed to separate the head 10 from the handle 20, similarly to the clip on a bicycle helmet.

The cooling device 100 also includes an electrical interface 30. The electrical interface 30 includes two transceivers 30a, 30b, where one transceiver is disposed in the head 10 and the other is disposed in the handle 20. The electrical interface 30 provides a means for the head 10 to transmit temperature information from the temperature sensor 22 to the handle 20. Moreover, the electrical interface 30 provides a means to transfer power from a power source in the handle 20 to the cooling element 24 in the head 10.

The electrical interface 30 is a wired interface having a plug and socket type configuration. In other embodiments, the electrical interface 30 is a wireless transmitter, where magnetic coils are used to transfer electrical energy across a gap by inducing current.

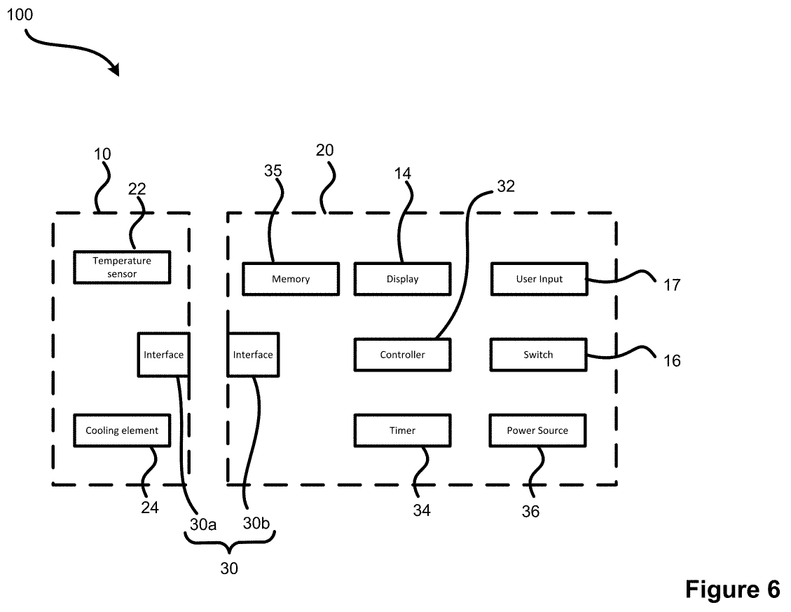

Further internal components of the handle 20 are will now be described with reference to FIG. 6. A controller 32 such as a microprocessor is used to determine the length of time for which to active the cooling element 24. This is based on the initial temperature of the tissue or organ surface and the body part around which the tissue or organ surface is disposed. Alternatively, the time is pre-set. The time is set so that the tissue or organ surface is cooled quickly, but not so quickly as to cause shock. The length of time that the cooling element 24 has been in operation is determined from a timer 34. The timer 34 may be integrated with the controller 32.

The controller 32 also determines the amount of the current to supply to the cooling element 24 in order for the cooling element to be set at a particular temperature, known as the cooling temperature. The controller 32 can control the current by adjusting a variable resistor or a rheostat disposed between a power source 36 and the cooling element 24. The cooling temperature can be varied while cooling is taking place, until the temperature of the tissue or organ surface reaches a target temperature. The cooling temperature is optimised based on the cooling time and initial surface temperature such that the tissue or organ surface is effectively cooled without the patient feeling initial shock as the device wo makes contact with the tissue or organ surface.

The memory 35 stores program instructions for allowing the controller 32 to function. For example, the memory 35 may be include a lookup table for associating tissue or organ surface temperatures with duration of cooling element 24 operation and cooling temperature.

The handle 20 also includes a power source 36 for driving the cooling element 24 in the head 10. The power source 36 according to some embodiments is a battery pack. In other embodiments, the power source 36 is a convertor for converting a mains power supply into a DC voltage for driving the cooling element 24.

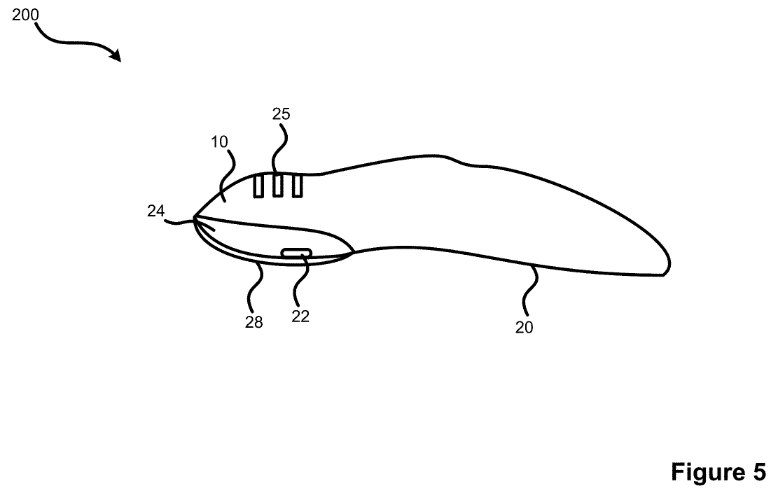

FIG. 5 shows a cooling device 200 according to another embodiment. Here, the head 10 and handle 20 are integrally formed. To achieve the necessary degree of sterilisation, a sterile pad 28 is adhered to the distal end of the cooling device 200 at the region that will make contact with the tissue or organ surface. The sterile pad 28 may adhere to the cooling device 200 using an adhesive. Alternatively, the sterile pad 28 may be held in place using, for example, static electricity, Velcro™, or a magnet.

The controller 32 is preprogramed with the thickness of the sterile pad 28, and is therefore able to compute the tissue or organ surface temperature using the temperature measured at the temperature sensor 22 which is separated from the tissue or organ surface by the sterile pad 28. Additionally, by being preprogramed with the thickness of the sterile pad 28, the controller 32 can calculate the cooling temperature required at the cooling element 24 to achieve the desired tissue or organ surface target temperature. In other embodiments, the user can select whether or not a sterile pad 28 is used, or specify the thickness of the sterile pad 28, using the user input 17.

The sterile pad 28 is made of a disposable material, and the cooling device 200 can be reused by attaching another sterile pad 28 to the device 200. The sterile pad 28 is non-porous, so that bacteria and liquids on the tissue or organ surface cannot soak through the sterile pad 28 and make contact with the lower surface of the cooling element 24.

The distal end of the cooling device 200 may take the shape of the heads 10 described with reference to FIGS. 1, 2 and 3. The sterile pad 28 may be cut by the user or pre-shaped to match the shape of the distal end of the cooling device 200.

FIG. 7 shows a perspective view of a cooling element 24 according to an embodiment. The cooling element 24 is a thermoelectric device which uses the Peltier effect. The thermoelectric device may, for example, be a thermocycler, single stage, or multistage device. The thermoelectric device is a sandwich structure, with a first conductive material 40 being covered on both sides by a second conductive material 38. A positive electrode 42 and a negative electrode 44 protrude from one of the layers of second material 38 for receiving current from the power source 36 in the handle 20.

When two conductors are placed in electric contact, electrons flow out of the one in which the electrons are less bound, into the one where the electrons are more bound. The reason for this is a difference in the so-called Fermi level between the two conductors. The Fermi level represents the demarcation in energy within the conduction band of a metal, between the energy levels occupied by electrons and those that are unoccupied.

When two conductors with different Fermi levels make contact, electrons flow from the conductor with the higher level, until the change in electrostatic potential brings the two Fermi levels to the same value. Current passing across the junction results in either a forward or reverse bias, resulting in a temperature gradient. If the temperature of the hotter junction (heat sink) is kept low by removing the generated heat, the temperature of the cold plate can be cooled by tens of degrees.

The first conductive material 40 is an array of N- and P-type semiconductors. The thermoelectric semiconductor material most often used is an alloy of Bismuth Telluride (Bi₂Te₃) that has been suitably doped to provide individual blocks or elements having distinct “N” and “P” characteristics. Other thermoelectric materials include Lead Telluride (PbTe), Silicon Germanium (SiGe), and Bismuth-Antimony (Bi—Sb) alloys. The second conductive material 38 is for example a copper plate or an aluminium plate. Although not shown, ceramic plates may be disposed on the outside surface of at least one of the second conductive material 38 plates.

FIG. 8 is a flowchart illustrating a method of cooling a tissue or organ surface using is the cooling device 100. In a first optional step Shoo, the controller 32 receives an indication of the body part into which the user intends to inject. For example, the controller 32 may receive “thighs” through the user input 17.

The temperature sensor 22 then measures the initial tissue or organ surface temperature in step S610, and provides the temperature information corresponding to the temperature to the controller 32. Step 610 outlines just one example of receiving an initial surface temperature. In other embodiments, the initial tissue or organ surface temperature is assumed using a lookup table. Here, a storage means stores a list of tissues and organs and their corresponding normal temperature. The controller then looks up the necessary tissue or organ initial temperature when the tissue or organ is input by the user. For example, in a healthy human the skin surface temperature is about 34 degrees Celsius. In still other embodiments, the user can input the initial surface temperature manually using the user input 17.

In step S620, the controller 32 determines the cooling temperature based on the initial temperature of the tissue or organ surface, the target temperature and optionally the selected body part. The target temperature is the tissue or organ surface temperature required to numb the nerve endings, which may be disposed around 2.5 millimetres below the dermis, without causing damage to the tissue or organ surface. The cooling temperature is the temperature of the cooling element, which may be colder than or the same as the target temperature. The colder the cooling temperature, the quicker the tissue or organ surface reaches the target temperature. However, when working with humans and animals, it is not desirable to place an item that is too cold on a tissue or organ surface, or cool the tissue or organ surface too quickly, as this may shock the patient. Therefore, in some embodiments the controller 32 optimises the cooling time and cooling temperature according to the initial tissue or organ surface temperature, target temperature and optionally the selected body part. The target temperature is typically between −10 degrees Celsius and −5 degrees Celsius. However, in some specific cases the target temperature can be between −30 degrees Celsius and 15 degrees Celsius.

In step S630, the controller 32 controls the current provided to the cooling element 32 in order to set the cooling temperature. After the calculated time period, the controller 32 automatically turns off the cooling element 24.

In other embodiments, the tissue or organ surface temperature is measured at predetermined intervals, such as once per second, and the controller 32 turns off the cooling element 24 when the target temperature is reached.

Advantages of the cooling device 100, 200 of the invention reside in reducing the discomfort caused by piercing a tissue or organ surface (internal or external) with a needle or scalpel or the like. Moreover, the cooling device advantageously comprises a disposable element, so that the whole device does not need to be replaced between uses. Furthermore, by having interchangeable heads, the cooling device can be made bespoke for different areas of the body.

1. A cooling device for cooling a subject's tissue or organ surface, the device comprising:
a handle;
a head comprising:
an electrical cooling element for cooling the subject's tissue or organ surface; and
a controller configured to:
receive an initial temperature of the subject's tissue or organ surface;
determine a cooling temperature based on the initial temperature of the subject's tissue or organ surface and a target temperature; and
control the cooling element to generate the cooling temperature.

2. The cooling device according to claim 1, comprising an interface for coupling the head to the handle.

3. The cooling device according to claim 2, comprising a fixing means for removably attaching the head to the handle, optionally wherein the fixing means comprises a locking mechanism for fixing the angle of the head with respect to the handle such that the applying pressure to the handle applies pressure to the head and/or the fixing means comprises a hook disposed on one of the head and the handle and a groove disposed on the other one of the head and the handle.

4. (canceled)

5. (canceled)

6. The cooling device according to claim 1, wherein the controller is disposed in the handle; and/or the head is integrally formed with the handle, and wherein the head comprises a removable non-porous layer between the cooling element and the subject's tissue or organ surface, optionally wherein the non-porous layer comprises an adhesive.

7. (canceled)

8. (canceled)

9. The cooling device according to claim 1, wherein the head is rotatable with respect to the handle, such that the head can contact the subject's tissue or organ surface while the handle is held away from the subject's tissue or organ surface, optionally wherein the head is rotatable from about −90° to about +90° to the longitudinal axis of the handle.

10. (canceled)

11. The cooling device according to claim 1, wherein the controller is configured to determine a cooling time based on the initial temperature of the subject's tissue or organ surface, the target temperature, and the cooling temperature, and/or the shape of the cooling element substantially corresponds to the shape of the head.

12. The cooling device according to claim 1, comprising:
a temperature sensor for measuring the temperature of the subject's tissue or organ surface; and/or
at least one fin for dissipating heat generated by the cooling element; and/or
a window in the cooling element for allowing light to pass through the head to the subject's tissue or organ surface, optionally wherein the window comprises a transparent material or an air gap.

13. (canceled)

14. (canceled)

15. The cooling device according to claim 1, wherein the head is annular and defines an aperture, through which a medical instrument can be passed or the head comprises one or more spaced-apart curvilinear arms which mutually define an open-ended aperture.

16. (canceled)

17. (canceled)

18. (canceled)

19. (canceled)

20. The cooling device according to claim 1, wherein the cooling element is configured to cool a subject's tissue or organ surface, when activated, to a target temperature of between −30° C. and 15° C., or between −15° C. and 0° C., or between −10° C. and −5° C.

21. The cooling device according to claim 1, wherein the cooling element comprises a thermoelectric device using the Peltier effect, optionally wherein layers of the thermoelectric device comprise bismuth and copper.

22. (canceled)

23. The cooling device according to claim 1, wherein the upper surface of the head comprises at least one vent for dissipating heat generated by the cooling element, and/or wherein at least the lower surface of the head is fabricated from an expandable material adapted to expand and contract with thermal expansion and/or contraction of the cooling element, and/or the lower surface of the head is fabricated from a highly thermally conductive material to maximize thermal energy transfer from the cooling element to the subject's tissue or organ surface to which it is applied, and/or the lower surface of the head is one of concave, convex or a combination of the two.

24. (canceled)

25. (canceled)

26. (canceled)

27. The cooling device according to claim 1, wherein the head defines a hollow cylinder, and wherein the cooling element is disposed on the outer surface of the hollow cylinder such that the inside of a cylindrically-shaped vessel can be cooled, optionally wherein the inner surface of the hollow cylinder comprises at least one vent for dissipating heat generated by the cooling element.

28. (canceled)

29. The cooling device according to claim 1, wherein the head is disposable and/or comprises a removable sterile cover.

30. (canceled)

31. The cooling device according to claim 1, wherein the controller is configured to:
vary the cooling temperature; and/or
turn off the cooling element when the target temperature is reached; and/or
calculate a cooling time, and turn off the cooling element after the cooling time; and/or
optimize the cooling temperature based on the cooling time, temperature of the subject's tissue or organ surface, and target temperature.

32. (canceled)

33. (canceled)

34. The cooling device according to claim 1, comprising a user input for selecting a subject's body part to be cooled, and the controller is configured to determine the cooling temperature based on the selected body part.

35. (canceled)

36. The cooling device according to claim 1, wherein the handle comprises:
a power source; and/or
a display for displaying at least one of: the remaining time before the target temperature is reached, the current temperature of the subject's tissue or organ surface, and an indication of whether the target temperature is reached; and/or
an alarm for indicating whether the target temperature is reached; and/or
a thermally insulating material; and/or
a catheter.

37. (canceled)

38. (canceled)

39. (canceled)

40. (canceled)

41. A head for a cooling device for cooling a subject's tissue or organ surface, the head comprising:
an electrical cooling element for cooling a subject's tissue or organ surface; and
an interface for coupling the head to a handle of a cooling device, such that the head can communicate with the handle.

42. (canceled)

43. A handle for a cooling device for cooling a subject's tissue or organ surface, the handle comprising:
an interface for coupling the handle to a head of the cooling device, such that the handle can communicate with the head; and
a controller configured to:
receive an initial temperature of the subject's tissue or organ surface;
determine a cooling temperature based on the initial temperature of the subject's tissue or organ surface and a target temperature; and
control the cooling element to generate the cooling temperature.

44. (canceled)

45. A method of cooling a subject's tissue or organ surface, the method comprising:
receiving the initial temperature of the subject's tissue or organ surface;
determining a cooling temperature based on the initial temperature of the subject's tissue or organ surface and a target temperature; and
controlling a cooling element to generate the cooling temperature.

46. The method according to claim 45, wherein the initial temperature of the subject's tissue or organ surface is received from a temperature sensor, optionally wherein the initial temperature of the subject's tissue or organ surface is received from a storage means.

47. (canceled)

48. (canceled)

49. The method according to claim 45, comprising:
varying the cooling temperature; and/or
receiving an indication of a subject's body part to be cooled, and the determining a cooling temperature comprises determining a cooling temperature based on the initial temperature of the subject's tissue or organ surface, the target temperature and the body part; and/or
determining a cooling time and optimizing the cooling temperature based on the cooling time, initial temperature of the subject's tissue or organ surface, and target temperature; and/or
turning off the cooling element when the target temperature is reached or turning off the cooling element after the cooling element has been activated for the duration of the cooling time.

50. (canceled)

51. (canceled)

52. (canceled)

53. (canceled)
